(19) United States
(12) Patent Application Publication
(10) Pub. No.: US 2026/0124182 A1
(43) Pub. Date: May 7, 2026

(54) NITAZOXANIDE FOR THE TREATMENT OF HEPATIC IMPAIRMENT

(71) Applicant: GENFIT, LOOS (FR)

(72) Inventors: VANESSA LEGRY, EMMERIN (FR); PHILIPPE DELATAILLE, VILLENEUVE D'ASCQ (FR); SIMON DEBAECKER, PONT-A-MARCQ (FR); RÉMY HANF, LILLE (FR)

(21) Appl. No.: 19/118,054

(22) PCT Filed: Oct. 3, 2023
(86) PCT No.: PCT/EP2023/077363

§ 371 Date: Apr. 3, 2025

(30) Foreign Application Priority Data
Oct. 4, 2022 (EP) 22306479.1

(51) Int. Cl. A61K 31/426; A61K 31/7048; A61P 1/16

(52) U.S. Cl. CPC A61K 31/426; A61K 31/7048; A61P 1/16

(57) ABSTRACT

The present invention relates to a compound selected from nitazoxanide, tizoxanide, tizoxanide glucuronide and pharmaceutically acceptable salts thereof, for use in a method for the treatment of hepatic impairment.

### FIELD OF THE INVENTION

The present invention relates to a compound selected from nitazoxanide, tizoxanide, tizoxanide glucuronide and pharmaceutically acceptable salts thereof, for use in a method for the treatment of hepatic impairment.

### BACKGROUND OF THE INVENTION

Liver is one of the vital organs that play essential roles in metabolism. The functions of liver are mainly classified into circulatory function, excretory function, metabolic function, defense-detox function, and hematological function. When any of them is damaged, various characteristic symptoms of hepatopathy, such as feeling of fatigue, feeling of worthlessness, anorexia, jaundice, and low fever, will appear. Prolonged hepatic function disorders will cause illnesses such as hepatitis, hepatic cirrhosis, and hepatic carcinoma. Moreover, the liver is involved in the clearance of drugs via a variety of mechanisms and pathways (e.g., cytochrome P450 [CYP] enzyme pathways, glucuronidation, biliary excretion) and hepatic impairment/dysfunction/disease/disorders can alter the clearance of drugs. In addition, when a subject has liver impairment, toxic substances that are normally removed by the liver accumulate in the blood, thereby impairing the function of the brain.

Therefore, the provision of therapeutic strategies for the treatment of hepatic impairment is of paramount importance.

### SUMMARY OF THE INVENTION

The present invention relates to a compound selected from nitazoxanide (NTZ), tizoxanide (TZ), tizoxanide glucuronide (TZG) and pharmaceutically acceptable salts thereof, for use in a method for the treatment of hepatic impairment.

In a particular embodiment, the subject has mild, moderate or severe hepatic impairment according to the Child-Pugh score.

In another particular embodiment, the subject has nonalcoholic steatohepatitis (NASH) and hepatic impairment. In particular, the subject may have NASH and moderate or severe hepatic impairment.

In yet another embodiment, the compound is nitazoxanide. In a further embodiment, nitazoxanide is for oral administration, in particular formulated in a tablet. In yet another embodiment, the subject is administered a tablet comprising 500 mg nitazoxanide twice daily.

### BRIEF DESCRIPTION OF THE DRAWINGS

FIG. 1 is a set of photographs of picrosirius Red Fast Green-stained liver sections from a rat model of bile duct ligation (BDL) with LPS-induced liver impairment, receiving either NTZ or vehicle.

FIG. 2 is a set of graphs showing the effect of NTZ of the level of transaminases in plasma in a BDL+LPS-induced liver impairment rat model.

FIG. 3 is a set of graphs showing the effect of NTZ of hepatic function marker in a BDL+LPS-induced liver impairment rat model.

FIG. 4 is a graph showing the effect of NTZ on LPS-induced increase of urea.

FIG. 5 is a set of graphs showing the effect of NTZ on LPS-induced circulating cytokines.

FIG. 6 is a graph showing the effect of NTZ on LPS-induced brain edema.

### DETAILED DESCRIPTION OF THE INVENTION

Hepatic impairment or liver impairment is a condition wherein normal functioning of the liver is reduced. Liver impairment and liver failure are different conditions. Liver impairment refers to a condition in which the liver function is compromised but not completely lost. It may imply a partial reduction in liver function, which may manifest as elevated liver enzymes, mild jaundice, or other mild symptoms. Liver failure, such as acute liver failure and acute-on-chronic liver failure, is a much more severe condition where the liver loses its ability to function. Liver failure results in a severe and often life-threatening disruption of liver function, with significant biochemical abnormalities and multiple organ system involvement. The prognosis for liver impairment is generally more favorable, especially if the underlying cause is identified and managed promptly, whereas liver failure is a critical medical emergency with a high risk of mortality.

It is herein provided therapeutic strategies to treat hepatic impairment. More specifically, the present invention relates to a compound selected from nitazoxanide (NTZ), tizoxanide (TZ), tizoxanide glucuronide (TZG) and pharmaceutically acceptable salts thereof, for use in a method for the treatment of hepatic impairment.

In a particular embodiment, the compound is selected from NTZ, TZ and pharmaceutically acceptable salts thereof. In a further embodiment, the compound is selected from NTZ and pharmaceutically acceptable salts thereof. In yet another embodiment, the compound is NTZ.

In the context of the present invention, a therapeutically effective amount of the compound is administered to the subject. A “therapeutically effective amount” refers to an amount of the drug effective to achieve a desired therapeutic result. A therapeutically effective amount of a drug may vary according to factors such as the disease state, age, sex, and weight of the individual, and the ability of drug to elicit a desired response in the individual. A therapeutically effective amount is also one in which any toxic or detrimental effects of agent are outweighed by the therapeutically beneficial effects. The effective dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

The frequency and/or dose relative to the administration can be adapted by one of ordinary skill in the art, in function of the patient, the pathology, the form of administration, etc. Typically, the compound can be administered at a dose comprised between 0.01 mg/day to 4000 mg/day, such as from 50 mg/day to 2000 mg/day, such as from 100 mg/day to 2000 mg/day; and particularly from 100 mg/day to 1000 mg/day. In a particular embodiment, the compound is administered at a dose of about 1000 mg/day, in particular at 1000 mg/day. In a particular embodiment, the compound is administered orally at a dose of about 1000 mg/day, in particular at 1000 mg/day, in particular as a tablet. Administration can be performed daily or even several times per day, if necessary. In one embodiment, the compound is administered at least once a day, such as once a day, twice a day, or three times a day. In a particular embodiment, the compound is administered once or twice a day. In particular, oral administration may be performed once a day, during a meal, for example during breakfast, lunch or dinner, by taking a tablet comprising the compound at a dose of about 1000 mg, in particular at a dose of 1000 mg. In another embodiment, a tablet is orally administered twice a day, such as by administering a first tablet comprising the compound at a dose of about 400 mg, about 500 mg or about 600 mg, in particular at a dose of 500 mg, during one meal, and administering a second tablet comprising the compound at a dose of about 500 mg, in particular at a dose of 500 mg, during another meal the same day.

The compound as used in the present invention can be formulated in a pharmaceutical composition further comprising one or several pharmaceutically acceptable excipients or vehicles (e.g. saline solutions, physiological solutions, isotonic solutions, etc.), compatible with pharmaceutical usage and well-known by one of ordinary skill in the art. These compositions can also further comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc.

These compositions can be formulated in the form of injectable suspensions, syrups, gels, oils, ointments, pills, tablets, suppositories, powders, gel caps, capsules, aerosols, etc., eventually by means of galenic forms or devices assuring a prolonged and/or slow release. For this kind of formulations, agents such as cellulose, carbonates or starches can advantageously be used.

NTZ, TZ or TZG can be in the form of pharmaceutically acceptable salts particularly acid or base salts compatible with pharmaceutical use. Salts of NTZ, TZ and TZG include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound.

The compound for use according to the present invention may be administered by different routes and in different forms. For example, the compound(s) may be administered via a systemic way, per os, parenterally, by inhalation, by nasal spray, by nasal instillation, or by injection, such as for example intravenously, by intramuscular route, by subcutaneous route, by transdermal route, by topical route, by intra-arterial route, etc. Of course, the route of administration will be adapted to the form of the drug according to procedures well known by those skilled in the art.

In a particular embodiment, the compound is formulated as a tablet. In another particular embodiment, the compound is administered orally. In another particular embodiment, the compound is NTZ formulated in a tablet for oral administration. In yet another particular embodiment, the tablet comprises from 400 to 600 mg NTZ, more particularly 500 mg of NTZ.

In a particular embodiment, the compound is for use as a single active ingredient for the treatment of hepatic impairment. In yet another embodiment, the compound is for use in combination with another active ingredient, such as another compound useful in the treatment of hepatic impairment or useful in the treatment of at least one symptom of hepatic impairment or useful in the treatment of a condition which has caused or will cause hepatic impairment, such as a condition selected from hepatic infection, liver fibrosis, liver cirrhosis, nonalcoholic fatty liver disease or nonalcoholic steatohepatitis.

The compound for use according to the invention is administered to treat hepatic impairment in a subject in need thereof. The term “subject” as used herein refers to a mammal, preferably a human subject.

The Child-Pugh classification is the most widely used and is one way of categorizing hepatic function. Using this classification, the subjects are grouped on the basis of two clinical features (encephalopathy and ascites) and three laboratory-based parameters (S-albumin, S-bilirubin and prothrombin time). Hepatic dysfunction is categorized into groups called A, B and C, or “Mild”, “Moderate” and “Severe”, corresponding to 5-6, 7-9 and 10-15 scores, respectively. In a particular embodiment, the subject has mild, moderate or severe hepatic impairment, as assessed by Child-Pugh scoring system, which is well-known in the art. In another particular embodiment, the subject has moderate or severe hepatic impairment, more particularly severe hepatic impairment. In a further particular embodiment, the subject has nonalcoholic steatohepatitis (NASH) and hepatic impairment. In yet another embodiment, the subject has NASH and moderate or severe hepatic impairment. In a further embodiment, the subject has nonalcoholic steatohepatitis and severe hepatic impairment.

The term “treatment”, as used herein, relates to both therapeutic measures and prophylactic or preventative measures, wherein the goal is to prevent or slow down (lessen) an undesired physiological change or disorder. Beneficial or desired clinical results include, but are not limited to, alleviation of symptoms, stabilizing pathological state (specifically not worsening), slowing down or stopping the progression of the disease, improving or mitigating the pathological state. Particularly, for the purpose of the present invention, treatment is directed to slow the progression of hepatic impairment and reduce the risk of further complications. It can also involve prolonging survival in comparison with the expected survival if the treatment is not received.

In a particular embodiment, the compound is used to reduce the mortality associated to hepatic impairment. The compound can also be used to slow or stop the progression of hepatic impairment. In particular, the compound can be used to prevent, slow down, or stop the progression of hepatic impairment, in particular to prevent, slow down, or stop the progression to severe hepatic impairment in a subject suffering from mild or moderate hepatic impairment, in particular from moderate hepatic impairment. In particular, the compound can be used to reverse hepatic impairment from severe to moderate or mild hepatic impairment. In yet another particular embodiment, the compound can be used to reverse hepatic impairment from moderate to mild hepatic impairment.

### EXAMPLES

### Example 1: Evaluation of NTZ Efficacy on BDL+LPS-Induced Liver Impairment in Rats

### Materials and Methods

The objective of the study was to assess the efficacy of NTZ to prevent LPS-induced inflammation, hepatic dysfunction and brain edema in fibrotic rats that underwent BDL surgery. BDL surgery was performed on 30 Sprague Dawley rats (Janvier). After anesthesia, a median laparotomy was performed to expose the liver and duodenum. The main bile duct was identified and dissected. After that, the bile duct was ligated in two parts: a first ligation was made in the middle of the bile duct, and the second ligation was made above the entrance of the pancreatic duct. The bile duct was then cut in the middle, to avoid recanalization. Fourteen days after surgery, a blood sample was collected from the sublingual vein to measure markers of hepatic injury (serum aspartate aminotransferase (AST), total bile acids and bilirubin) to stratify the animals into treatment groups. Two rats with BDL were excluded at the end of the study for abnormal blood parameters (10-20% unsuccessful surgery expected in this model). Two unoperated animals were included in the study as healthy controls.

Twenty/twenty-one days after BDL surgery, hepatic impairment was induced by intraperitoneal administration of 1 μg/kg LPS (Escherichia coli O111:B4). Nine rats with BDL received phosphate buffer saline (PBS, Fisher Scientific, USA) instead of LPS to be used as BDL controls.

The body weight and food intake were monitored twice a week all along the study.

During the fibrosis induction phase, each animal was observed once a day for the monitoring of clinical signs. Observations include changes in the skin, fur, eyes, occurrence of secretions and excretions and autonomic activity (lachrymation, piloerection, unusual respiratory pattern). Changes in gait, posture, stereotypes (e.g. excessive grooming, repetitive circling) or bizarre behavior (self-mutilation, walking backward) are also monitored. Animals were euthanized in case of weight loss of more than 25% for more than 3 consecutive days, absence of food consumption, deterioration of the general condition or vocalization.

After LPS administration, each animal was continuously observed (appearance of the animal—change in fur, skin color, the degree of activity, vigilance—response to a stimulus, opening of the eyelids, activity, gait and behaviour) during 7 hours in order to assess the degree of severity of the inflammatory response and pain.

Treatment with NTZ 100 mg/kg or vehicle (1% carboxymethylcellulose (C4888, Sigma-Aldrich), 0.1% tween 80 (P8074, Sigma-Aldrich)) was administered by gavage 1 hour before LPS injection.

TABLE 1
Main products used

| Usual name or | | Supplier | Supplier batch | |
|---|---|---|---|---|
| INN | Supplier | reference | No | Storage conditions |
| Nitazoxanide | Interchim | RQ550 | FN262291501 | −20° C. |
| Lipopolysaccharides | Sigma Aldrich | L2630-10 MG | #0000112732 | 2-8° C. |
| from | | | | |
| Escherichia coli | | | | |
| O111:B4 | | | | |
| Sodium | Sigma Aldrich | C4888-500 G | SLBN8656V | RT |
| carboxymethyl | | | | |
| cellulose (CMC) | | | | |
| TWEEN ® 80 | Sigma Aldrich | P8074-100 ML | BCBT3893 | RT |
| Isoflurin | Axience | 152678 | IO56-01 | RT |

NTZ was protected from light because of its light sensitiveness.

For oral gavage, NTZ powder was dissolved in CMC 1%, Tween 80 0.1% at the concentration of 10 mg/ml in an amber glass bottle, homogenized with a polytron and sonicated 10 seconds at a power of 10%. NTZ was kept under magnetic stirring until the administration to the rats at 10 ml/kg.

LPS solution was prepared under a microbiological safety cabinet, in order to have a sterile solution to inject to rats. The LPS solution was dissolved in phosphate buffer saline (PBS) at 0.5 μg/ml, aliquoted and frozen until the day of experiment. LPS was administered at 2 ml/kg to the rats.

All surviving animals were euthanized 3 h after LPS injection for plasma and tissues analyses. At the end of the study, by plasma level of hepatic function markers and cytokines, and brain edema have been evaluated.

### Results:

### Evaluation of Fibrosis Level

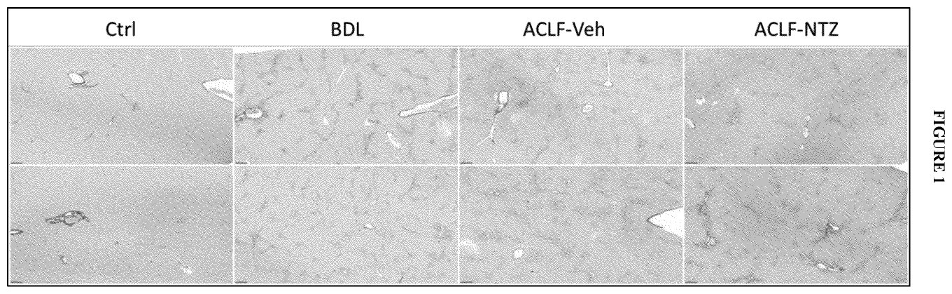

Peri-ductular fibrosis was observed in the liver of mice that underwent BDL surgery, as demonstrated by Sirius red staining (FIG. 1).

Neither LPS nor NTZ treatment affected the percentage of liver fibrosis (data not shown), as expected for such a short treatment.

### Plasma Levels of Hepatic and Renal Function Markers

BDL surgery induced severe liver injury and alterations of hepatic functions, that are further increased by LPS injection. The effect of NTZ on the plasma levels of hepatic and renal function markers was assessed.

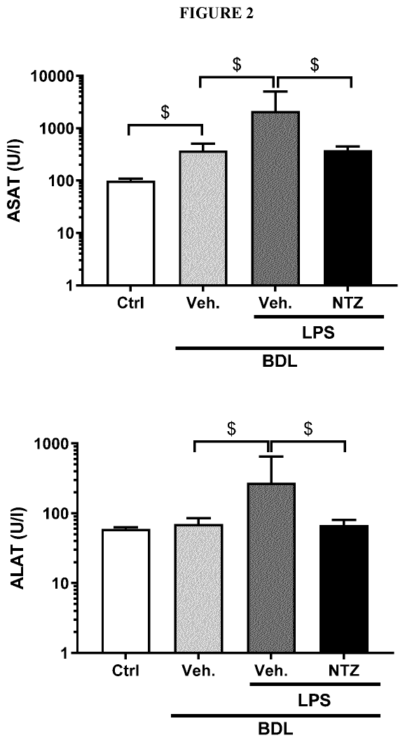

Plasma transaminases ALAT and ASAT, markers of hepatocellular injury, were increased in the BDL group compared to the non-pathological group. LPS further increased plasma level of ASAT and ALAT by 5.7 and 3.9 fold, respectively. NTZ treatment greatly reduced ALAT by 101% (p=0.03) and ASAT by 100% (p=0.01) compared to the ACLF-Veh condition (FIG. 2).

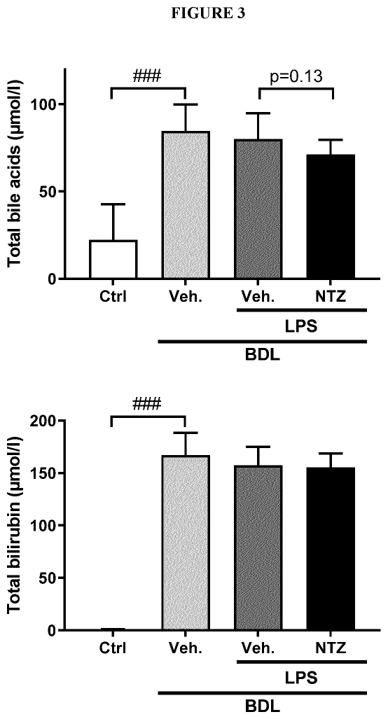

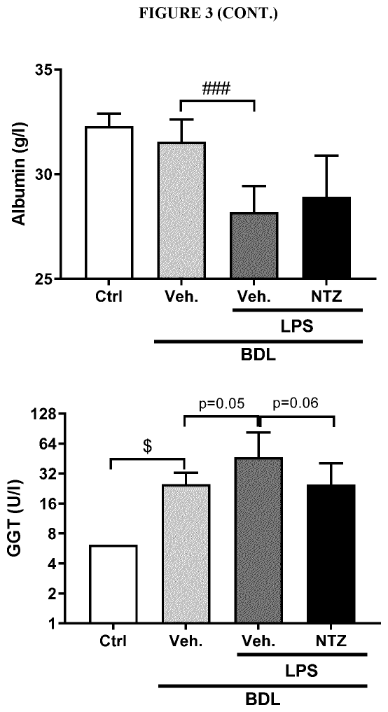

Plasma levels of total bile acids, total bilirubin, albumin and GGT, markers of hepatic functions, were also altered with LPS administration in BDL rats (FIG. 3). NTZ treatment tended to reduce the LPS-induced alteration of these markers

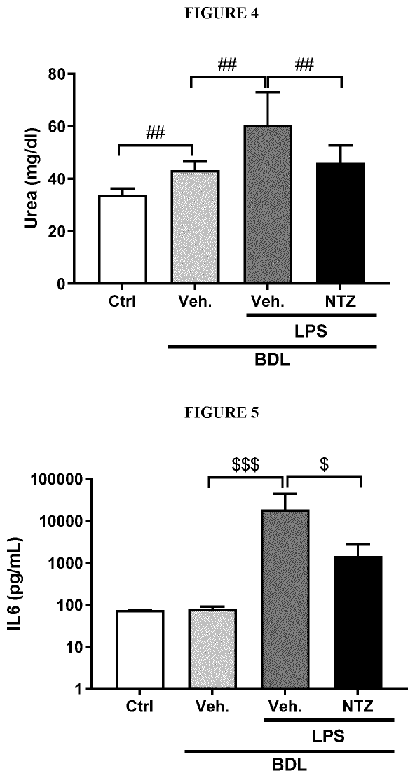

LPS injection also altered renal function as shown by elevated urea concentration (FIG. 4), confirming that organs other than the liver are affected in this model. NTZ treatment also prevented LPS-induced renal impairment by reducing plasma urea by 84%, p=0.007 (FIG. 4).

### Plasma Levels of Circulating Cytokines

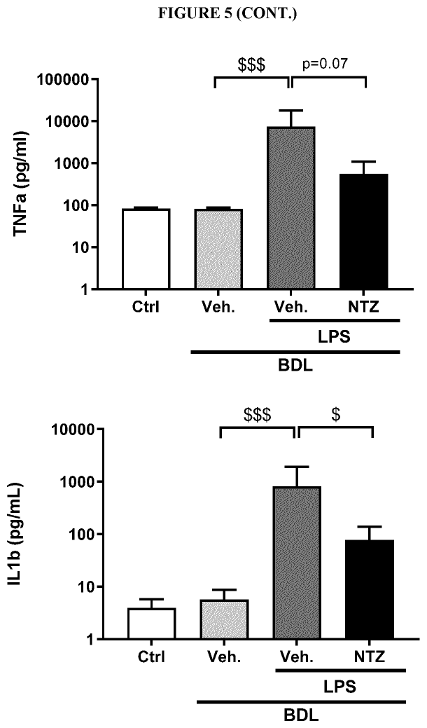

Circulating cytokines levels were measured in the animal blood collected 3 hours after LPS injection. While LPS injection induced a strong cytokines rise by 228 fold for IL6, 91 fold for TNFα, and 143 fold for IL-1β, NTZ decreased the level of circulating IL-6 by 93%, TNFα by 94%, and IL-1β by 91% (FIG. 5).

### Brain Edema

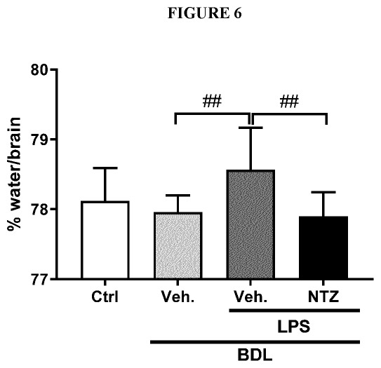

Finally, LPS injection induced brain edema (+0.78% in water content), while NTZ totally restored the water content to the level of BDL and healthy animals (−111% compared to the BDL condition, p=0.006) (FIG. 6).

### Conclusions:

BDL surgery induces severe liver injury and alterations of hepatic functions as seen with increased levels of AST (3.8-fold), total bile acids (3.8-fold), total bilirubin (165-fold), gamma-glutamyl transferase (GGT) (4-fold) and urea (1.3-fold) compared to healthy controls.

LPS injection in rats with BDL further increased AST (5.7-fold) and urea (1.4-fold) levels, while it also induced ALT increase (3.9-fold) and a drop in albumin concentration (−11%), compared to BDL rats.

NTZ treatment blocked the LPS-induced rise in AST (−100%), ALT (−101%) and urea (−84%). These effects were associated with a significant effect on systemic inflammation: while LPS injection induced a strong cytokines rise by 228 fold for IL6, 91 fold for TNFα, and 143 fold for IL-1β, NTZ decreased the level of circulating IL-6 by 93%, TNFα by 94%, and IL-1β by 91%. Brain edema (+0.78% in water content) was also induced in these animals, while NTZ totally restored the water content to the level of BDL and healthy rats.

Taken together, these results demonstrate the potency of NTZ to rapidly counteract systemic inflammation together with hepatic and brain damages induced by LPS in rats with hepatic impairment.

### Example 2: Design of a Study to Evaluate Nitazoxanide in the Treatment of Hepatic Impairment

The efficacy and safety of nitazoxanide is evaluated in patients with hepatic impairment.

A number of methods exist to categorize the severity of hepatic impairment. In the present study, the Child-Pugh classification (hereinafter “CP system”) has been selected since it is the most widely used and is an acceptable method supported by regulatory agencies, including the United States FDA and the European Medicines Evaluation Agency.

Hepatic impairment is classified as moderate or severe using the CP system. The parameters to determine the CP class for each subject with hepatic impairment will be collected at screening.

TABLE 2
Child-Pugh scoring parameters

| | Points scored for | | |
|---|---|---|---|
| | observed findings | | |
| | 1 | 2 | 3 |
| Hepatic | 0 | 1 or 2 | 3 or 4^{c} |
| encephalopathy | | | or subject on |
| grade^{a} | | | medication to |
| | | | prevent |
| | | | encephalopathy |
| Ascites^{b} | Absent | Slight | Moderate or |
| | | | subject on |
| | | | medication to |
| | | | control |
| | | | ascites |
| Serum bilirubin, | <2 (<34) | 2 to 3 | >3 (>50) |
| mg/dl (μmol/L) | | (34 to 50) | |
| Serum albumin, | >3.5 | 2.8 to 3.5 | <2.8 (>28) |
| g/dl (g/L) | (>35) | (28 to 35) | |
| Prothrombin | <4 | 4 to 6 | >6 |
| time, sec | | | |
| prolonged | | | |

Chronic hepatic impairment is classified into Child-Pugh (CP) class A to C, employing the added score of the 5 parameters in the above table. Mild Impairment (CP-A): 5 or 6 points; Moderate Impairment (CP-B): 7 to 9 points; Severe Impairment (CP-C): 10 to 15 points.
a In this study, hepatic encephalopathy is graded according to the following criteria:
Grade 0: normal consciousness, personality, neurological examination, or normal electroencephalogram;
Grade 1: restless, sleep disturbed, irritable/agitated, tremor, impaired handwriting, or 5 cycles per second (cps) waves;
Grade 2: lethargic, time-disoriented, inappropriate, asterixis, ataxia, or slow triphasic waves;
Grade 3: somnolent, stuporous, place-disoriented, hyperactive reflexes, rigidity, or slower waves;
Grade 4: unarousable coma, no personality/behavior, decerebrate, or slow 2 to 3 cps delta activity.
b Ascites is graded according to the following criteria:
Absent: No ascites is detectable by manual examination or by ultrasound investigation, if ultrasound investigation is performed;
Slight: Ascites palpitation doubtful, but ascites measurable by ultrasound investigation, if performed;
Moderate: Ascites detectable by palpitation and by ultrasound investigation, if performed;
Severe: Necessity of paracentesis; does not respond to medication treatment;
Subjects with a history of severe ascites who are receiving diuretics such as furosemide to prevent recurrence should receive the point score for the original degree of ascites.
c A subject with hepatic encephalopathy of Grade 3 or 4 would not be admitted into the study. However, a subject with a history of Grade 3 or 4 who is receiving a medication (Iactulose, neomycin, or rifaximin) to prevent recurrence of encephalopathy can be admitted into the study and will receive the point score for the stage 3 or 4 encephalopathy.

Each patient enrolled is administered a tablet containing 500 mg nitazoxanide twice daily.

### Example 3: Safety of Nitazoxanide in Patients with Hepatic Impairment

The study design was an open-label, nonrandomized, 2-center, multiple-dose, parallel-group study to evaluate the PK as well as the safety and tolerability of NTZ 500 mg BID administration for 7 days in males and females patients with moderate and severe hepatic impairment as compared to matched control healthy males and females patients with normal hepatic function.

The objective was to assess the effect of hepatic impairment on the PK of tizoxanide (TZ; NTZ active metabolite), following repeated oral dose administration of NTZ 500 mg BID for 7 days in subjects with moderate or severe hepatic impairment and subjects with normal hepatic function.

The following parameters used to measure the hepatic function were considered for the evaluation of the degree of hepatic impairment on NTZ PK: Child-Pugh (CP) score (*), serum albumin, serum total bilirubin, international normalized ratio (INR), aspartate aminotransferase (AST), alanine aminotransferase (ALT).

(*) For the control match healthy subject group, CP score was not calculated, and the analysis was limited to hepatic impaired patients.

Subjects were enrolled within the following groups based on their CP score at Screening:
Group 1: Normal hepatic function
Group 2: Moderate hepatic impairment (CP Class B)
Group 3: Severe hepatic impairment (CP Class C)

A total of 25 subjects were included in the study and received at least one dose of NTZ including 10 healthy subjects assigned to the control group, 9 subjects assigned to the moderate hepatic impairment group and 6 subjects assigned to the hepatic renal impairment group).

On Day 1, the study subjects took 500 mg NTZ (1 tablet) with 240 mL (8 fluid ounces) of water 30 minutes after the start of the standardized breakfast. No food was allowed for at least 4 h after each dose. Blood PK samples were taken at the following timepoints: pre-dose (completed in fasted state prior to the start of the standardized breakfast), T1h, T2h, T3h, T4h, T5h, T6h, T7h, T8h, T10h and T12h post-dose.

From Day 2 to Day 6, subjects received 500 mg of nitazoxanide (1 tablet) in the morning and in the evening for 6 consecutive days and on Day 7 the last dose was administered in the morning. Pre-dose blood PK sample was collected in fasted state prior to the start of the standardized breakfast each morning from Day 2 to Day 6.

On Day 7, blood PK samples were taken at the following timepoints: Pre-dose (completed in fasted state prior to the start of the standardized breakfast), T1h, T2h, T3h, T4h, T5h, T6h, T7h, T8h, T10h, T12h, T14h and T16h post-dose. PK samples at T18h, T24h and T48h post dose were taken respectively on D8 and D9.

Primary analysis for total TZ geometric mean for impaired and control groups and geometric mean ratios (GMR) (90% CI) with control group at steady state is presented in Table 3.

TABLE 3
Primary analysis: total tizoxanide geometric means for impaired and
control groups and geometric mean ratios (90% CI) with
control group at steady state

| | | Hepatic | | | |
|---|---|---|---|---|---|
| | | Impairment | | GM Ratio | |
| Analyte | Parameter | Group | GM | (Impaired/Control) | GM Ratio 90% CI |
| Total | AUC₀₋₁₂ | Control | 161740.6 | — | |
| TZ | (h* | Moderate (CP B) | 144921.1 | 0.8960 | (0.6169; 1.3014) |
| | nmol/L) | Severe (CP C) | 264488.5 | 1.6353 | (1.0805; 2.4748) |
| | AUC₀₋ₜ | Control | 165415.8 | — | |
| | (h* | Moderate (CP B) | 149925.4 | 0.9064 | (0.6184; 1.3283) |
| | nmol/L) | Severe (CP C) | 296755.0 | 1.7940 | (1.1739; 2.7417) |
| | Cₘₐₓ | Control | 35257.63 | — | |
| | (nmol/L) | Moderate (CP B) | 28235.09 | 0.8008 | (0.5469; 1.1726) |
| | | Severe (CP C) | 42031.87 | 1.1921 | (0.7871; 1.8056) |

The primary analysis was to assess the difference in the PK parameters (Cₘₐₓ, AUC₀₋₁₂ and AUC₀₋ₜ) of total TZ after repeated oral administration of NTZ 500 mg BID for 7 days in subjects with moderate or severe hepatic impairment and subjects with normal hepatic function.

For subjects with moderate hepatic impairment, no statistically significant difference with the control group was elicited for AUCs and Cₘₐₓ. Given that, the GMR were between 0.80 and 1.00, a difference for subjects with moderate hepatic impairment is unlikely even if not formally demonstrated due to the limited sample size.

For subjects with severe hepatic impairment, statistically significant differences were elicited with the control group for AUCs. GMR were between 1.64 and 1.79 and their 90% CI excluded 1.00. The GMR for Cₘₐₓ was estimated at 1.19 and not statistically different from the control group.

No difference could be elicited on total TZ parameters between subjects with moderate hepatic impairment and control subjects, as well as on Cₘₐₓ for subjects with severe hepatic impairment. However, AUCs were moderately increased for subjects with severe hepatic impairment. The increases were statistically significant.

Secondary analysis for total TZ accumulation ratios is presented in Table 4.

TABLE 4
Secondary analysis: total tizoxanide accumulation ratios

| | | Hepatic | | | GM | GM |
|---|---|---|---|---|---|---|
| | | Impairment | | | Ratio | Ratio |
| Analyte | Parameter | Group | Visit | GM | (Day 7/Day 1) | 90% CI |
| Total | AUC₀₋₁₂ (h*nmol/L) | Total | DAY 1 | 114685.7 | — | |
| TZ | | | DAY 7 | 183262.0 | 1.5980 | (1.4499; 1.7611) |
| | Cₘₐₓ (nmol/L) | Total | DAY 1 | 24810.88 | — | |
| | | | DAY 7 | 34716.85 | 1.3993 | (1.2625; 1.5508) |

Group by Day interaction term was not significant, hence ratios were computed for groups combined

The secondary analysis was to determine the accumulation ratios on AUC₀₋₁₂ and Cₘₐₓ measured after a single (Day 1) and a 7-day repeated (BID) oral administrations.

No interaction between Day and group factors was evidenced for any parameter. Therefore, the accumulation was assumed independent of the degree of hepatic impairment and was assessed for all groups combined.

For total TZ, statistically significant accumulations with limited magnitude were evidenced for Cₘₐₓ and AUC₀₋₁₂. GMR between Day 7 and Day 1 was around 1.4 for Cₘₐₓ and around 1.6 for AUC₀₋₁₂.

The sensitivity analyses confirmed that there is probably no effect of moderate hepatic impairment on total TZ PK. Severe hepatic impairment also seemed to have no impact on Cₘₐₓ but might increase the AUC by about 1.5-fold.

The relationship between hepatic function markers (serum albumin, serum total bilirubin, INR, AST and ALT (obtained at screening)) and PK parameters (Cₘₐₓ and AUC₀₋₁₂) for TZ and TZ Glu (total and unbound) were investigated and evaluated using graphical and correlation techniques.

Both Pearson (supporting a linear trend) and Spearman correlation (supporting a monotonic trend) coefficients were calculated. The correlation coefficients (p) were used to measure appropriately the association for most possible relationship and only strong correlations (i.e. p≥0.75) would support further modeling.

The summary of the relationship between hepatic function and PK parameters is presented in Table 5. Table 1

TABLE 5

| Analyte | Parameter | AST | ALT | Albumin | Bilirubin | INR |
|---|---|---|---|---|---|---|
| Tizoxanide | AUC₀₋₁₂ | P (NS): 0.17 | P (LS): −0.07 | P (NS): −0.4 | P (NS): 0.43 | P (NS): 0.46 |
| | (h · nmol/L) | | | | | |
| | Cₘₐₓ | P (LS): −0.05 | P (LS): −0.17 | P (NS): −0.25 | P (NS): 0.47 | P (NS): 0.46 |
| | (nmol/L) | | | | | |
| Tizoxanide | AUC₀₋₁₂ | P (LS): −0.12 | P (LS): −0.08 | S (NS): −0.22 | S (NS): 0.15 | S (NS): −0.11 |
| glucuronide | (h · nmol/L) | | | | | |
| | Cₘₐₓ | P (LS): −0.27 | P (LS): −0.17 | P (LS): 0.18 | P (LS): −0.09 | S (NS): −0.3 |
| | (nmol/L) | | | | | |
| u-Tizoxanide | AUC₀₋₁₂ | P (NS): 0.15 | P (LS): −0.06 | P (NS): −0.37 | P (NS): 0.47 | P (NS): 0.5 |
| | (h · nmol/L) | | | | | |
| | Cₘₐₓ | S (NS): −0.09 | P (LS): −0.15 | P (NS): −0.24 | P (NS): 0.51 | P (NS): 0.49 |
| | (nmol/L) | | | | | |
| u-Tizoxanide | AUC₀₋₁₂ | P (NS): 0.12 | S (NS): −0.07 | P (NS): −0.36 | P (NS): 0.29 | P (NS): 0.1 |
| glucuronide | (h · nmol/L) | | | | | |
| | Cₘₐₓ | P (LS): −0.14 | P (LS): −0.16 | S (NS): −0.21 | S (NS): 0.29 | S (NS): −0.2 |
| | (nmol/L) | | | | | |

P: Pearson correlation coefficient
S: Spearman correlation coefficient
NS: Natural scale
LS: Log-scale
Font: Red: strong correlation
Orange: Moderate correlation
Green: Weak correlation
Black: Poor correlation

Table 1 Summary of correlations between PK parameters and markers of hepatic function—highest coefficients among Pearson and Spearman coefficients, calculated on natural and log scales

The results showed that for most markers of hepatic function, the correlation with TZ or TZ Glu (total or unbound) PK parameters were poor (p<0.25) or weak (0.25≤p<0.5). An isolated moderate linear (p=0.51) correlation was identified between unbound TZ Cₘₐₓ and bilirubin, but the correlation was too weak (p<0.75) to support further modeling.

For tizoxanide, time to reach maximum concentration was consistent between groups and days (median tₘₐₓ between 3.5 and 5.0 h). With moderate to high inter-individual variability, the exposure of total TZ was higher in severe hepatic impairment group than other groups on Day 1 and Day 7 (GM ratios of Cₘₐₓ, AUC₀₋₁₂ and AUC_{0-∞} around 0.8- to 1.0-fold between moderate and control groups, and around 1.1- to 1.8-fold between severe and control groups).

In all subjects and regardless of the day, AUC_{0-∞}, was determined with a percentage of extrapolation below 20%.

The elimination phase was comparable between groups on Day 1 but seemed slower on Day 7 in severely hepatic impaired subjects (GM t_{1/2} around 2 h on Day 1 vs. GM t_{1/2} of 4 h on Day 7 for severe impairment group). Trough concentrations overall appeared to increase with the level of hepatic impairment.

In urine, the fraction of the dose recovered over 12 h on both Day 1 and Day 7 was less than 1% regardless of the group and had high inter-individual variability. The renal clearance was also low in all groups regardless of the day, with values ranging from 0.002 to 0.030 L/h and high inter-individual variability.

Statistical analysis (primary analysis) on peak exposure (Cₘₐₓ), area under the curve from time zero to 12 h (AUC₀₋₁₂) and from time zero to the time of last quantifiable concentration (AUC₀₋ₜ) could not elicit a difference in subjects with moderate hepatic impairment and control subjects, with GMRs within 0.80-1.25 suggesting the absence of relevant differences. In severe hepatic impairment group, statistically significant differences were elicited for AUCs only, with GMR between 1.64 and 1.79.

The sensitivity analyses for total TZ were consistent with the primary analysis. The trends observed for PK parameters were consistent and they confirmed that there probably is no effect of moderate hepatic impairment on total TZ's PK. However, severely impaired subjects also seemed to have no impact on Cₘₐₓ but might increase the AUC around 1.5-fold. Secondary analysis showed a statistically significant accumulations for Cₘₐₓ and AUC₀₋₁₂, with ratios between Day 7 and Day 1 around 1.4 for Cₘₐₓ and 1.6 for AUC₀₋₁₂.

This clinical trial shows that nitazoxanide is safe and metabolized into its active metabolite.

### Example 4: Efficacy of Nitazoxanide in Patients with Hepatic Impairment

This study is conducted to evaluate the efficacy of nitazoxanide 500 mg twice daily in patients with hepatic impairment. The treatment is anticipated to improve the subject's condition in view of the results reported in example 1.

This is an interventional, proof-of-concept, phase 2a, randomized, open-label, controlled, parallel-group clinical study to evaluate the safety, pharmacokinetics, and efficacy of nitazoxanide (NTZ) 500 mg twice daily (BID) administered orally on a background of standard of care (SOC) versus SOC alone in patients with acute-on-chronic liver failure (ACLF).

The study will consist of 3 periods: a 72-hour screening period, followed by a 7-day treatment period, and a safety follow-up period up to 90 days from the start of treatment.

Patients will be randomized to receive nitazoxanide 500 mg twice daily (BID) orally on a background of SOC or SOC alone for 7 days.

Standard of Care (SOC) is defined as the standard medical management of patients with ACLF as per local practices at the participating clinical sites. Medical treatment at the site is to be guided by the treating physician, taking into account his/her overall clinical assessment of the patient, precipitating event(s), and the nature and severity of organ dysfunction. Interventions may include (but are not limited to) the supportive management with fluid therapy, including albumin infusion where indicated, antibiotics for suspected or confirmed infections, nutrition, bowel management, thromboprophylaxis, and organ system support, including cardiovascular support, oxygen and non-invasive or invasive respiratory support, laxatives and non-absorbable antibiotics for hepatic encephalopathy, and dialysis for renal failure. SOC may also include liver transplantation as deemed necessary for individual patient clinical condition and per local institutional practices.

The primary objective is to evaluate the safety of nitazoxanide (NTZ) in patients with ACLF as hepatic impairment.

The secondary objectives were:
To evaluate the pharmacokinetics of the main metabolites of NTZ (tizoxanide and tizoxanide glucuronide) in patients with ACLF
To evaluate the effect of NTZ on clinical disease progression using applicable prognostic criteria in patients with ACLF
To evaluate the effect of NTZ on clinical outcomes, including liver transplant, 28-Day and 90-Day mortality

Other objectives were to evaluate the effect of NTZ therapy on inflammatory markers in these patients, to evaluate the effect of NTZ on the intestinal microbiota.

Approximately 30 patients will be randomized in a 2:1 ratio (NTZ+SOC:SOC) in this study as follows:
Arm A: NTZ+SOC.
Arm B: SOC

Safety information, clinical prognostic scores, markers of renal and hepatic function, intestinal microbiota profile and inflammatory markers will be assessed as described in the schedule of assessment at screening, Day 1 through Day 7 (active treatment phase), Day 8, Day 14, Day 21, Day 28, and Day 90 (follow-up visits).

PK measurements of NTZ and metabolites will be assessed on Day 1, Day 7, and Day 8 (NTZ+SOC arm only).

Clinical outcomes, including liver transplantation and mortality, will be monitored throughout the study and up to Day 97.

### Exclusion Criteria:

### Patients Presenting any of the Following Exclusion Criteria Will not be Included in the Trial:

1) Patients with cirrhosis who develop decompensation at any time in the post-operative period following partial liver resection or major non-liver surgery
2) Patients with uncontrolled infection or sepsis. Patients with infection may be entered into the study provided antimicrobials have been administered for at least 48 hours with an appropriate response observed prior to randomization as deemed acceptable by the principal investigator.
3) Patients with ACLF grade 3
4) Patients with MELD-Na score >25
5) Hepatic encephalopathy grade III (stupor) or IV (coma) in accordance with West Haven criteria
6) Patients with clinical evidence of disseminated intravascular coagulation
7) Patients who require the use of inotropic support because of cardiovascular dysfunction
8) Patients with evidence of significant and/or uncontrolled bleeding as judged by the principal investigator
9) Patients on mechanical ventilation or expected to require mechanical ventilation
10) Patients with active malignancy and (with expected survival of less than one year) or history of malignancies other than hepatocellular carcinoma (HCC) within Milan criteria or curatively treated skin cancer (basal cell or squamous cell carcinomas), unless adequately treated or in complete remission for five or more years
11) Known hypersensitivity to nitazoxanide, or nitazoxanide use within 30 days before screening
12) Hemoglobin <9 g/dl
13) All patients on dialysis
14) Patients currently on warfarin. Prior use of warfarin permitted provided no use within 5 days prior to start of screening.
15) Treatment with systemic corticosteroid therapy within 30 days of screening
16) Participation in another interventional trial (drug or device) or use of other investigational product within 30 days of screening
17) Pre-existing gastrointestinal disease that alters intestinal absorption (such as coeliac disease, inflammatory bowel disease, etc.)
18) Significant systemic or major illness other than liver disease, including coronary artery disease, cerebrovascular disease, pulmonary disease, renal failure, serious psychiatric disease, that, in the opinion of the Investigator would preclude the patient from participating in and completing the study
19) Pregnant or lactating females or females planning to become pregnant during the study period
20) Positive anti-human immunodeficiency virus (HIV) antibody
21) Individuals for whom the investigator deems that study participation would be unsafe
22) Patients on the UNOS liver transplant list
23) Positive illicit drug test (except for marijuana) unless the subject uses any of these drugs as prescriptions and is approved by the Investigator
24) Positive alcoholic test except for patients with alcoholic hepatitis
25) History of significant drug abuse within 1 year prior to screening or recreational use of soft drugs (except for marijuana) within 1 month or hard drugs (such as cocaine, phencyclidine [PCP], opioid derivatives including heroin, and amphetamine derivatives) within 3 months prior to screening.

### Criteria for Evaluation:

### Primary Endpoint:

The primary endpoint is the incidence of treatment emergent adverse events (TEAEs), serious adverse events (SAEs), and treatment discontinuation and for safety related reasons up to 90 days.

### Secondary Endpoints:

### Safety Endpoints:

a. Laboratory measurements (chemistry, hematology and coagulation)
b. Physical examinations, vital signs, and electrocardiogram (ECG)

### Pharmacokinetic Endpoints:

The following PK parameters expressed in terms of unbound as well as total concentrations will be determined when applicable for TZ and TZ-glucuronide after the single dose oral administration (Day 1) and at steady state (projected steady state reached by Day 7):
maximum observed plasma concentration (Cₘₐₓ),
area under the plasma concentration-time curve (AUC) from time zero to 12h (AUC0-12),
AUC from time zero to the time of the last quantifiable concentration (AUC0-t),
time of the maximum observed plasma concentration (Tₘₐₓ),
apparent plasma terminal elimination half-life (t_{1/2}).

### Secondary Efficacy Endpoints:

Change from baseline in: MELD-Na, CLIF-C ACLF scores, CLIF-C organ failure score, ACLF grade, Child-Turcotte-Pugh (CTP) score, West Haven criteria.
Clinical outcomes—any other hepatic clinical event such as ascites, GI bleeding, liver transplant, 28-day and 90-day mortality.

1-11. (canceled)

12. A method of treating hepatic impairment comprising administering a compound selected from nitazoxanide (NTZ), tizoxanide (TZ), tizoxanide glucuronide (TZG) and pharmaceutically acceptable salts thereof to a subject having hepatic impairment.

13. The method according to claim 12, wherein the subject has mild hepatic impairment according to the Child-Pugh score.

14. The method according to claim 12, wherein the subject has moderate hepatic impairment according to the Child-Pugh score.

15. The method according to claim 12, wherein the subject has severe hepatic impairment according to the Child-Pugh score.

16. The method according to claim 12, wherein the subject has nonalcoholic steatohepatitis and hepatic impairment.

17. The method according to claim 16, wherein the subject has NASH and moderate hepatic impairment.

18. The method according to claim 16, wherein the subject has NASH and severe hepatic impairment.

19. The method according to claim 12, wherein the compound is nitazoxanide.

20. The method according to claim 12, wherein nitazoxanide is administered orally.

21. The method according to claim 20, wherein nitazoxanide is formulated in a tablet.

22. The method according to claim 21, wherein the subject is administered tablet comprising 500 mg nitazoxanide twice daily.
